(19) United States
(12) Patent Application Publication
(10) Pub. No.: US 2026/0367040 A1
(43) Pub. Date: Nov. 19, 2026

(54) DEVICE AND METHOD FOR DETECTING A DEFECT IN AN OBJECT CONTAINING WIRE ROPES

(71) Applicant: COMPAGNIE GENERALE DES ETABLISSEMENTS MICHELIN, Clermont-Ferrand (FR)

(72) Inventors: AURELIEN MONTOY, Clermont-Ferrand (FR); LOUIS MARGUERIN, Clermont-Ferrand (FR)

(21) Appl. No.: 19/135,658

(22) PCT Filed: Dec. 5, 2023
(86) PCT No.: PCT/FR2023/051935

§ 371 Date: Jun. 4, 2025

(30) Foreign Application Priority Data
Dec. 5, 2022 (FR) FR2212779

(51) Int. Cl. G01N 27/82; G01N 33/2045

(52) U.S. Cl. CPC G01N 27/82; G01N 33/2045

(57) ABSTRACT

A detection device and method (1) detect, in an object containing a plurality of metal cords, a positioning defect of one of the metal cords relative to another of the metal cords. The device (1) comprises: a magnetic field generator (10) arranged to generate an inspection magnetic field and to be positioned facing the object so that the plurality of metal cords causes the deformation of the inspection magnetic field, and a primary magnetic sensor (12) arranged to measure the intensity of the inspection magnetic field, The inspection magnetic field has, before being deformed by the plurality of metal cords, at least one neutral point at which the intensity of the inspection magnetic field is zero, and the primary magnetic sensor (12) is placed relative to the magnetic field generator (10) in a chosen position that corresponds to a position of this neutral point.

### FIELD OF THE INVENTION

The present invention relates to the field of detecting a defect in an object containing metal cords, and more specifically a positioning defect of one metal cord relative to another metal cord and, yet more particularly, detecting a defect in an object forming part of a tyre such as a reinforcing ply containing a plurality of reinforcing metal cords embedded in a rubber-based matrix.

### PRIOR ART

During the manufacturing of a tyre, the metal carcass ply is wrapped around a thin liner and two edges of the carcass ply are welded to each other so as to give the carcass ply a cylindrical shape.

However, the weld can have defects that cause irreversible quality problems once the tyre curing step has been performed. Poor welding of the carcass ply can cause the destruction of the tyre when running or, to a lesser extent, premature wear of the tyre or discomfort when running. As a result, the weld must be checked following the laying of the carcass ply.

Currently, this check can be performed using different methods:
a metal comb is used to determine the position and spacing of the edges in the weld. This method is not necessarily reliable and requires the intervention of an operator to handle the comb;
a laser profilometer is used to scan the carcass ply before and after welding. This method is costly due to the necessary hardware and dedicated software for analysing the defects.

There is not therefore currently a solution for checking the welding of carcass plies, and more generally reinforcing plies, during tyre manufacturing, that is low-cost and easy to use.

### SUMMARY OF THE INVENTION

One aim of the invention is to provide a solution for detecting defects in an object containing a plurality of metal cords, such as a metal carcass ply of a tyre, that is low-cost, easy to implement, reliable and preferably automated.

According to a first aspect, a detection device is proposed for detecting, in an object containing a plurality of metal cords, such as a reinforcing ply containing a plurality of reinforcing metal cords embedded in a rubber-based matrix, a positioning defect of one of the metal cords relative to another of said metal cords, said device comprising:
a magnetic field generator, preferably consisting of a magnet or a coil, that is arranged to generate a magnetic field referred to as the “inspection magnetic field”, and to be positioned facing the object so that the plurality of metal cords causes the deformation of said inspection magnetic field,
a magnetic sensor referred to as the “primary magnetic sensor” that is arranged to measure the intensity of the inspection magnetic field,
   said device being characterized in that the inspection magnetic field has, before being deformed by the plurality of metal cords, at least one neutral point at which the intensity of said inspection magnetic field is zero,
   and in that the primary magnetic sensor is placed relative to the magnetic field generator in a chosen position that corresponds to a position of this neutral point.

According to advantageous, non-limiting features, taken individually or in any combination:
the magnetic field generator has a toroidal shape that is centred on an axis referred to as the “central axis of the generator” and defines a free inner space, and in that the adjustment system comprises a support part that holds the primary magnetic sensor and is engaged in said free inner space so that it can be moved along the central axis of the generator in order to modify the position of the primary magnetic sensor along said central axis of the generator;
the device comprises at least one stabilizing element, such as a roller, capable of coming into contact with the object in order to maintain a constant distance between the primary magnetic sensor and an outer surface of the object;
the device comprises at least one suspension member, such as a spring, capable of elastically pressing the stabilizing element against the object;
the device comprises a secondary magnetic sensor that is separate from the primary magnetic sensor, is not subject to the inspection magnetic field, and is arranged to be placed facing a zone of the object in order to measure the intensity of the magnetic field known as the “background magnetic field”, which is representative of a magnetic field prevailing in the object in the absence of the inspection magnetic field;
the device comprises a sub-assembly forming a train of three carriages that are placed in series in a direction referred to as the “longitudinal direction” and are articulated in pairs, one relative to the other, so that they are able to follow an outer surface of the object, each bearing on said outer surface in order to provide at least three corresponding separate bearing zones along the longitudinal direction, said carriages respectively being made up of:
a primary carriage that holds the magnetic field generator and the primary magnetic sensor, together with at least one primary stabilizing element, preferably a primary roller the axis of rotation of which is perpendicular to the longitudinal direction, which allows said primary carriage to roll on the outer surface of the object while maintaining the magnetic field generator and the primary magnetic sensor at a constant, predefined distance from the outer surface,
a secondary carriage that holds the secondary magnetic sensor and at least one secondary stabilizing element, preferably a secondary roller the axis of rotation of which is perpendicular to the longitudinal direction, which allows said secondary carriage to roll on the outer surface while maintaining the secondary magnetic sensor at a constant, predefined distance from the outer surface,
a tertiary carriage that holds at least one tertiary stabilizing element, preferably a tertiary roller the axis of rotation of which is perpendicular to the longitudinal direction, which allows said tertiary carriage to roll on the outer surface.

According to another aspect, a detection assembly is proposed, characterized in that it comprises a detection device as set out above, together with a drive system making it possible to generate a relative travelling movement of an outer surface of the object with respect to the magnetic field generator and the primary magnetic sensor in a predetermined sense referred to as the “scanning sense”, and in that the secondary magnetic sensor is arranged so as to precede the primary magnetic sensor with respect to said scanning sense, so that the same zone of the outer surface of the object is covered successively by the secondary magnetic sensor, which measures the background magnetic field, and then by the primary magnetic sensor, which measures the inspection magnetic field as deformed by the plurality of metal cords of the object.

According to another aspect, a method is proposed for detecting, in an object containing a plurality of metal cords, such as a reinforcing ply containing a plurality of reinforcing metal cords embedded in a rubber-based matrix, a positioning defect of one of the metal cords relative to another of said metal cords, the method comprising the following steps:
a) supplying a magnetic field generator and a primary magnetic sensor, in order to form a detection device, the primary magnetic sensor being placed relative to the magnetic field generator in a chosen position that corresponds to the position of a neutral point, the neutral point corresponding to a point of an inspection magnetic field generated by the magnetic field generator, before it is deformed by the plurality of metal cords, at which the intensity of said inspection magnetic field is zero;
b) positioning the detection device relative to the object so that the magnetic field generator and the primary magnetic sensor are placed facing the object;
c) moving the detection device relative to the object and continuously acquiring a primary signal received by the primary magnetic sensor;
d) detecting, in the primary signal, a signature representative of a positioning defect of a metal cord of the object.

According to advantageous, non-limiting features, taken individually or in any combination:
step a) comprises a step a0) of adjusting the detection device by positioning the primary magnetic sensor relative to the magnetic field generator in the chosen position;
in step a0), the primary magnetic sensor (12) is positioned using an adjustment system (143) that makes it possible to adjust, for example by screwing, the position of the primary magnetic sensor (12) relative to the magnetic field generator (10) and to keep the primary magnetic sensor (12) in the chosen position, fixed relative to the magnetic field generator (10), the adjustment system (143) being contained in a support (14), referred to as the “primary support”, which holds the magnetic field generator (10) and the primary magnetic sensor (12) and is provided with the adjustment system (143);
the magnetic field generator (10) has a toroidal shape that is centred on an axis (A) referred to as the “central axis of the generator” and defines a free inner space (101), and the adjustment system (143) comprises a support part (142) that holds the primary magnetic sensor (12) and is engaged in said free inner space (101) and in step a0), the support part (142) holding the primary magnetic sensor (12) is moved along the central axis of the generator (A) in order to modify the position of the primary magnetic sensor (12) along said central axis of the generator (A);
in step b), at least one stabilizing element (18), such as a roller, is placed in contact with the object (2) in order to maintain a constant distance between the primary magnetic sensor (12) and an outer surface (22) of the object (2);
in step b), the stabilizing element (18) is elastically pressed against the object (2) by means of a suspension member (19), such as a spring;
during step c), the detection device is moved relative to the object so that the primary magnetic sensor is placed successively in different positions relative to the object, the different positions comprising at least one position of interest in which the magnetic sensor is placed facing a zone of interest of the object, in which a potential defect is to be detected, and a nominal position in which the magnetic sensor is placed facing a zone of the object without defects, different from the zone of interest, so that a nominal magnetic field intensity of the primary signal is measured;
the signature representative of a positioning defect of a metal cord of the object is a local extremum relative to the nominal magnetic field intensity, the local extremum having a magnetic field intensity greater than a first threshold value or a magnetic field intensity less than a second threshold value;
in step d), if the presence of a local extremum is determined and, if this extremum has a magnetic field intensity greater than the first threshold value, it is concluded that a defect is present corresponding to the superposition of two metal cords, whereas, if this extremum has a magnetic field intensity less than the second threshold value, it is concluded that a defect is present corresponding to the existence of a gap between two consecutive metal cords that is greater than a nominal gap;
the method comprises a step prior to acquiring the primary signal of acquiring a secondary signal corresponding to the change during the movement of a magnetic field known as the “background magnetic field”, which is representative of a magnetic field prevailing in the object in the absence of the inspection magnetic field and in which step d) comprises the processing of the primary signal including the subtraction of the secondary signal from the primary signal;
the prior step of acquiring a secondary signal is implemented by a secondary magnetic sensor (16) that is separate from the primary magnetic sensor (12) and is not subject to the inspection magnetic field and, during the implementation of the prior step of acquiring a secondary signal, the secondary magnetic sensor (16) is placed facing a zone of the object (2).
in step b), a sub-assembly (13) is placed in contact with an outer surface (22) of the object (2), the sub-assembly (13) forming a train of three carriages (130a, 130b, 130c) that are placed in series in a direction (D) referred to as the “longitudinal direction” and are articulated in pairs, one relative to the other, so that they are able to follow an outer surface (22) of the object (2), each bearing on said outer surface (22) in order to provide at least three corresponding separate bearing zones along the longitudinal direction (D), said carriages (130a, 130b, 130c) respectively being made up of:
a primary carriage (130a) that holds the magnetic field generator (10) and the primary magnetic sensor (12), together with at least one primary stabilizing element (18a), preferably a primary roller the axis of rotation (Xa) of which is perpendicular to the longitudinal direction (D), which allows said primary carriage (130a) to roll on the outer surface (22) of the object (2) while maintaining the magnetic field generator (10) and the primary magnetic sensor (12) at a constant, predefined distance from the outer surface (22),
a secondary carriage (130b) that holds the secondary magnetic sensor (16) and at least one secondary stabilizing element (18b), preferably a secondary roller the axis of rotation (Xb) of which is perpendicular to the longitudinal direction (D), which allows said secondary carriage (130b) to roll on the outer surface (22) while maintaining the secondary magnetic sensor (16) at a constant, predefined distance from the outer surface (22),
a tertiary carriage (130c) that holds at least one tertiary stabilizing element (18c), preferably a tertiary roller the axis of rotation (Xc) of which is perpendicular to the longitudinal direction (D), which allows said tertiary carriage (130c) to roll on the outer surface (22) of the object (2), and
in order to implement the prior step of acquiring a secondary signal and step c) of moving the detection device (1) relative to the object (2) and continuously acquiring a primary signal received by the primary magnetic sensor (12), the carriages (130a, 130b, 130c) are rolled on the outer surface (22) of the object (2) so that the same zone of the object (2) is first facing the primary carriage (130a), then the secondary carriage (130b) and finally the tertiary carriage (130c);
step d) comprises the processing of the primary signal, this processing comprising at least one processing operation among the following processing operations: filtering with a low-pass filter, calculation of the absolute value of the signal, normalization, thresholding, and a combination of these processing operations;
the method is implemented in order to detect, in a reinforcing ply containing metal cords extending non-parallel to a selvedge of the reinforcing ply, a positioning defect of one of the metal cords relative to another of said metal cords.

### DESCRIPTION OF THE FIGURES

Other features and advantages of the present invention will become apparent from reading the following description of a preferred embodiment. This description will be given with reference to the appended figures, in which:

FIG. 1 shows a defect detection device in use on an object;

FIG. 2 shows part of an object without any defects;

FIG. 3 shows part of an object having a cord gap defect;

FIG. 4 shows part of an object having a cord superposition defect;

FIG. 5 shows a support holding a primary magnetic sensor and a magnetic field generator;

FIG. 6 shows a magnetic field generator;

FIG. 7 shows a magnetic field generator and the inspection magnetic field;

FIG. 8 is a cross-sectional view of a support holding a primary magnetic sensor and a magnetic field generator;

FIG. 9 shows the device comprising three carriages including a primary carriage holding a magnetic field generator and a primary magnetic sensor;

FIG. 10 shows the steps of a defect detection method;

FIG. 11 shows a signal received by the primary magnetic sensor.

### DETAILED DESCRIPTION OF THE INVENTION

### Device

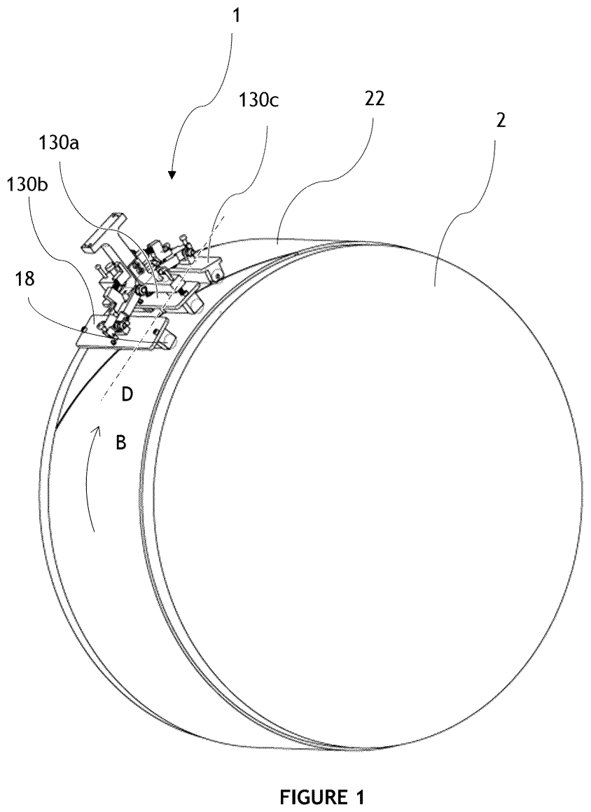

With reference to FIG. 1, a detection device 1 is proposed for detecting, in an object 2 containing a plurality of metal cords 20, a positioning defect of one of the metal cords relative to another of the metal cords.

Said metal cords 20 are preferably placed in a non-metal matrix 21, preferably an elastomer matrix, for example a rubber-based matrix.

The object 2 is for example a reinforcing ply containing a plurality of reinforcing metal cords embedded in a rubber-based matrix 21, such as a carcass ply or a crown ply of a tyre. The object 2 can therefore be a composite object comprising metal and rubber.

The object 2 can also be a bead wire for holding a tyre in position on a rim.

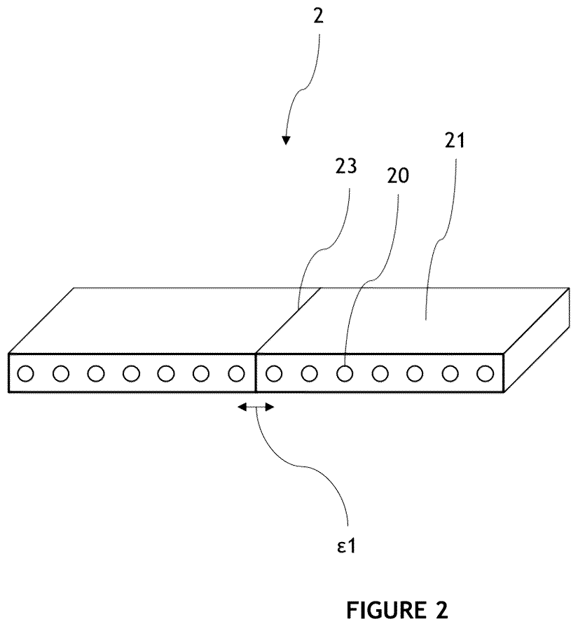

With reference to FIG. 2, the object 2 contains a plurality of metal cords 20 placed in a theoretical arrangement. For example, the cords 20 can be placed in a regular pattern, for example placed at equal distances from each other and parallel to each other. More specifically, as illustrated in FIG. 2, the cords 20 can be distributed at a predefined nominal spacing ε1, the spacing extending by the centre distance between neighbouring sections of cords. The cords can also be placed at different distances from each other. The cords 20 can be straight or undulating, for example.

The cords 20 preferably extend in a direction non-parallel to the selvedges of the object 2. In the case of a ply of a tyre, the selvedges of the ply usually denote the boundaries in terms of width, i.e. the edges that axially limit the ply, and that run parallel to each other and in the circumferential direction of the tyre. In the case of a carcass ply, the cords 20 are generally perpendicular to the selvedges of the ply. In the case of a crown ply, the cords 20 are generally oriented obliquely relative to the selvedges.

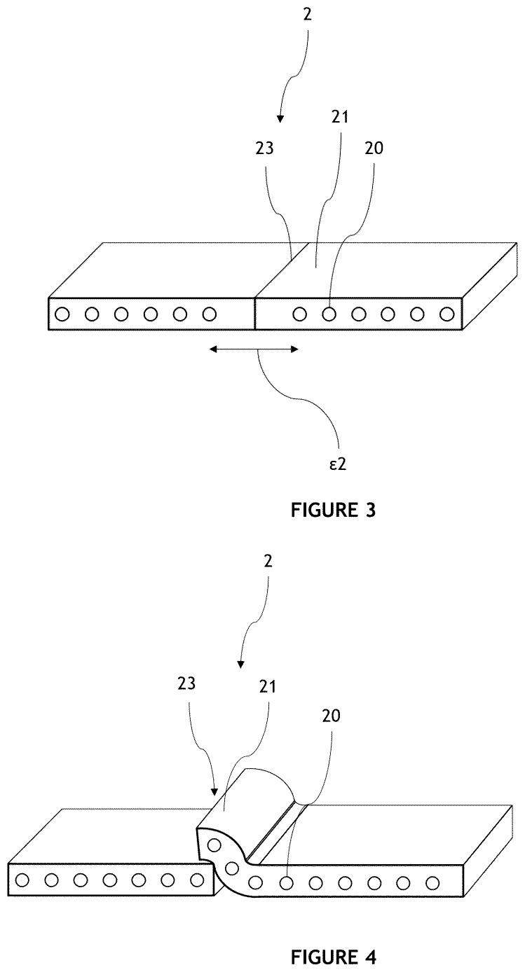

The object 2 can have a defect. More specifically, there can be a positioning defect of one of the metal cords 20 relative to another of the metal cords 20 of the object 2. As illustrated in FIG. 3, the defect can for example consist of a spacing ε2 between two consecutive metal cords 20 that is greater than an expected nominal gap between two consecutive metal cords 20. The defect can also consist of the superposition of two metal cords 20, as illustrated in FIG. 4.

More particularly, the object 2 can have a defect in the weld 23 between two edges of the object 2. The edges of the object 2 are sides of the object 2 that connect each of the selvedges of the object 2 together. The edges of the object 2 therefore extend in a direction non-parallel to the selvedges of the object 2. The edges preferably extend parallel to the cords 20, and the weld 23 thus typically extends parallel to the cords 20. In the case of a carcass ply, the edges, and thus the weld 23, are generally perpendicular to the selvedges of the ply. In the case of a crown ply, the edges, and thus the weld 23, are generally oriented obliquely relative to the selvedges. The weld 23 is a zone of the object 2 that can particularly have defects is the weld 23 is poorly performed. The weld 23 generally constitutes a zone of interest in the detection of defects of the object 2.

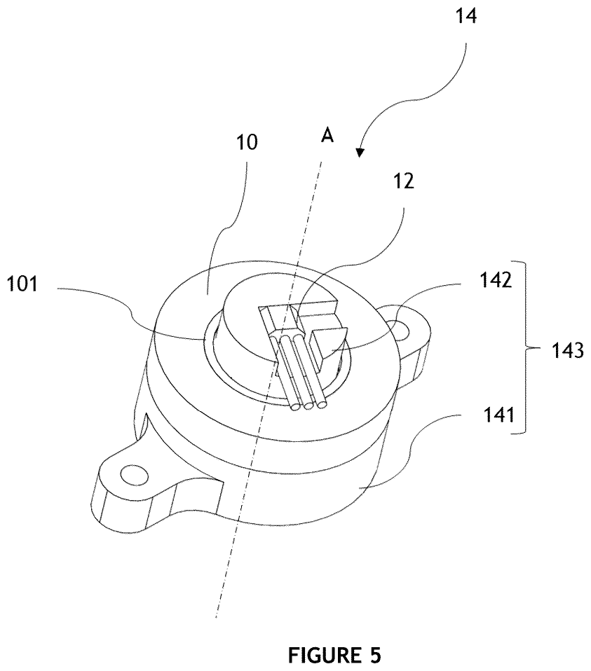

With reference to FIG. 5, the detection device 1 comprises a magnetic field generator 10. The magnetic field generator 10 is for example a magnet, an electromagnet or a coil.

Advantageously, the magnetic field generator 10 is a single element, that is, a single a magnet, a single electromagnet or a single coil. In other words, the magnetic field generator 10 is not made up of a combination of elements such as a combination of magnets.

The magnetic field generator 10 generates a magnetic field referred to as the “inspection magnetic field”. The coil has the advantage of allowing better control of the parameters of the magnetic field.

As illustrated in FIG. 5, the magnetic field generator 10 preferably has a toroidal shape that is centred on an axis A referred to as the “central axis of the generator” and defines a free inner space 101. It will be understood that the central axis of the generator A is the axis of rotation of the magnetic field generator 10. Preferably, the magnetic field generator 10 has the shape of a torus with a rectangular cross-section.

Preferably, the central axis of the generator A corresponds to the axis connecting the north pole and the south pole of the magnetic field generator 10.

The magnetic field generator 10 can have other shapes, preferably shapes exhibiting symmetry of revolution centred on a central axis A. The magnetic field generator 10 can for example be a hollow annular or toroidal cylinder with a circular cross-section.

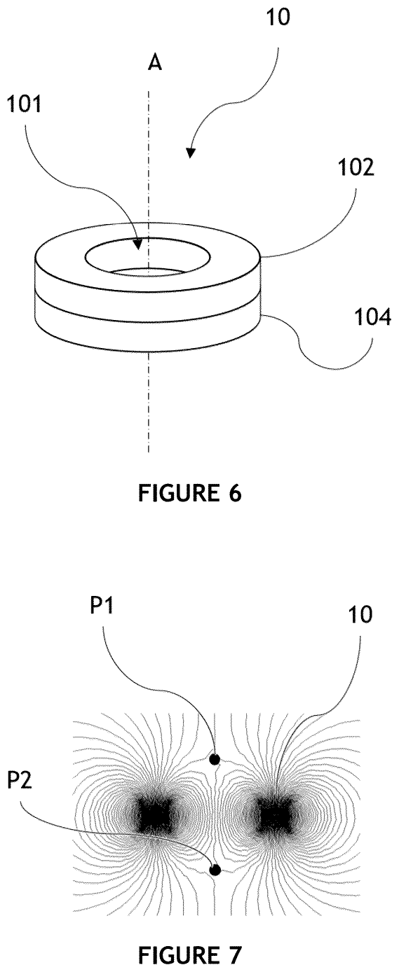

With reference to FIG. 6, the magnetic field generator 10 is designed so that the north and south poles are placed one on top of the other. The magnetic field generator 10 therefore comprises a north part 102 and a south part 104 and, in the example of a magnetic field generator 10 having a toroidal shape centred on a central axis A, each of the north part 102 and south part 104 has a toroidal shape centred on the central axis A.

When the inspection magnetic field is not subject to magnetic disturbances linked to metal elements (therefore, for example, when the magnetic field generator 10 is sufficiently far away from the metal cords 20 of the object 2 so that the inspection magnetic field is not deformed by said metal cords 20 of the object 2), the inspection magnetic field has at least one neutral point at which its intensity is zero. “Zero” is given to mean a magnetic field intensity the absolute value of which is preferably equal to 0 Tesla. It will be understood that a neutral point is an intrinsic point of the magnetic field of the magnetic field generator 10.

Preferably, the neutral point or points of the magnetic field of the magnetic field generator 10 are positioned on the axis connecting the north pole and the south pole of the magnetic field generator 10.

As illustrated in FIG. 7, at the neutral points P1 and P2, the inspection magnetic field lines substantially cancel each other out and the inspection field has a substantially zero intensity value. At a neutral point, the deformations of the lines of the inspection magnetic field are thus easy to detect as it must merely be detected that the magnetic field intensity is no longer zero, or is at least separated by a certain difference from a zero intensity value, at a neutral point, in order to deduce that the lines of the inspection magnetic field are deformed.

As illustrated in FIG. 5, the detection device 1 also comprises a magnetic sensor 12, referred to as the “primary magnetic sensor”. The primary magnetic sensor 12 is configured to acquire magnetic signals and therefore to measure magnetic field intensity values. More specifically, the primary magnetic sensor 12 is arranged to measure the intensity of the inspection magnetic field.

Preferably, the primary magnetic sensor 12 is a Hall effect sensor.

The primary magnetic sensor 12 is placed relative to the magnetic field generator 10 in a chosen position that corresponds to the position of a neutral point of the inspection magnetic field. Consequently, in the absence of magnetic disturbance able to lead to the deformation of the inspection magnetic field, the primary magnetic sensor 12 is assumed to measure a magnetic field intensity that is not influenced by the inspection magnetic field.

This primary magnetic sensor 12 is therefore arranged to measure a magnetic field intensity at a neutral point. The primary magnetic sensor 12 is thus arranged to detect variations in intensity of the inspection magnetic field at a neutral point and therefore to detect deformations of the inspection magnetic field.

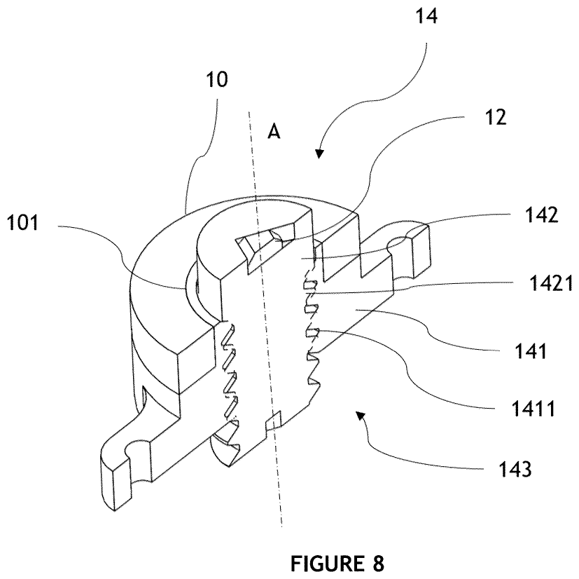

Preferably, with reference to FIGS. 5 and 8, the device 1 comprises a support 14 referred to as the “primary support”. The primary support 14 holds the magnetic field generator 10 and the primary magnetic sensor 12.

According to a certain embodiment, the primary support 14 comprises a first support part 141 to which the magnetic field generator 10 is fastened, and second support part 142 to which the primary magnetic sensor 12 is fastened.

The primary support 14 is preferably provided with an adjustment system 143 that makes it possible to adjust the position of the primary magnetic sensor 12 relative to the magnetic field generator 10.

For example, as illustrated in FIG. 8, the first support part 141 comprises a first thread 1411 and the second support part 142 comprises a second thread 1421 that complements the first thread 1411. The second support part 142 is capable of being screwed through the first support part 141 so that the position of the primary magnetic sensor 12 relative to the magnetic field generator 10 can be adjusted by screwing or unscrewing the second support part 142 screwed through the first support part 141. In this example, the adjustment system 143 is considered to comprise the first support part 141 and the second support part 142.

According to the embodiment illustrated in FIG. 8, in which the magnetic field generator 10 has a toroidal shape, it will be understood that the second support part 142 is engaged in the free inner space 101 delimited by the magnetic field generator 10 so that it can be moved along the central axis of the generator A, by screwing or unscrewing.

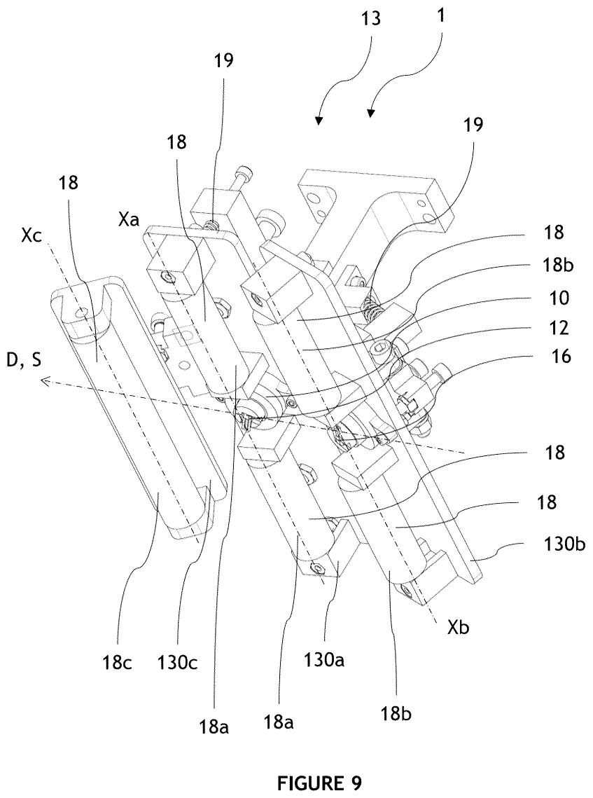

Preferably, as illustrated in FIG. 9, the device 1 comprises a secondary magnetic sensor 16 that is separate from the primary magnetic sensor 12. The secondary magnetic sensor 16 is intended to be arranged so that it is not subject to the inspection magnetic field. In other words, the secondary magnetic sensor 16 is sufficiently far away from the magnetic field generator 10 so that the intensity of the inspection magnetic field to which the secondary magnetic sensor 16 is subjected is zero. The secondary magnetic sensor 16 is thus arranged so that, when the device 1 is placed facing the object 2, the zone of the object 2 that is facing the secondary magnetic sensor 16 is not subject to the inspection magnetic field.

The secondary magnetic sensor 16 is arranged to measure the intensity of a magnetic field referred to as the “background magnetic field” representative of a residual magnetic field of the object 2, that is the magnetic field prevailing in the object 2 in the absence of the inspection magnetic field. This residual magnetic field can be due to the magnetization of the object 2, more specifically the magnetization of the metal cords of the object 2, for example following the placing of a magnet near the object 2 for a certain period. The background magnetic field can also be representative of a magnetic field of the environment of the device 1 and the object 2, that is for example the industrial environment, which can comprise different magnetic tools.

Preferably, as illustrated in FIG. 9, the device 1 comprises at least one stabilizing element 18 capable of coming into contact with the object 2 in order to maintain a constant, predefined distance between the magnetic field generator 10 and the object 2 and between the primary magnetic sensor 12 and the object 2 when the device 1 is in use. The predefined distance depends in particular on the size and density of the cords 20. It can for example be of the order of a few millimetres, for example 4 millimetres.

More specifically, as illustrated in FIG. 1, the stabilizing element 18 is capable of coming into contact with an outer surface 22 of the object 2 when the device 1 is in use. “In use” is given to mean that the device 1 is being used to detect a defect in an object 2. In use, it is desirable to maintain a predefined distance between the magnetic field generator 10 and the object 2 so that the object 2 is subject to the inspection magnetic field in a similar way in any zone of the object 2 opposite which the magnetic field generator 10 is positioned during use. Likewise, in use, it is desirable to maintain a predefined distance between the primary magnetic sensor 12 and the object 2 so that the primary magnetic sensor 12 takes measurements in similar conditions with respect to the object 2 during use.

According to the embodiment in which the device 1 comprises a secondary magnetic sensor 16, a stabilizing element 18 can also make it possible to maintain a constant, predefined distance between the secondary magnetic sensor 16 and the object 2 when the device 1 is in use.

The stabilizing element 18 is preferably capable of allowing the relative movement of the object 2 with respect to the device 1 while being in contact with the object 2. More particularly, preferably, the stabilizing element 18 is capable of rolling on the object 2.

The stabilizing element 18 can take the form of a roller as in the example illustrated in FIGS. 1 and 9. A roller makes it possible to control the direction of movement of the device 1 relative to the object 2. The roller also makes it possible to distribute the contact surface between the device 1 and the object 2 and to control the distance between the primary magnetic sensor 12 and the object 2 (and optionally also between the secondary magnetic sensor 16 and the object 2). The stabilizing element 18 can also be a load-bearing ball for example. The diameter of the rollers or balls can depend on the diameter of a drum around which the object 2 is stretched. The diameter of the rollers or balls can for example be a few centimetres, for example 2 centimetres.

Advantageously, with reference to FIG. 9, the device 1 comprises at least one suspension member 19 capable of elastically pressing the stabilizing element or elements 18 against the object 2 when the device 1 is in use. The suspension member 19 can for example comprise a spring.

The stabilizing element or elements 18 and the suspension member 19 can also make it possible to compensate for any inaccuracy of the positioning of the device 1 relative to the object 2 by a robotic arm.

According to a preferred embodiment illustrated in FIG. 9, the device 1 comprises a sub-assembly 13 forming a train of three carriages 130a, 130b, 130c placed in series in a direction D referred to as the “longitudinal direction”. According to this embodiment, the device comprises at least three stabilizing elements 18a, 18b, 18c.

As illustrated in FIG. 1, the three carriages 130a, 130b, 130c are articulated in pairs one relative to the other so that they are capable of following the outer surface 22 of the object 2, each bearing on the outer surface 22. When the device 1 is in use and each carriage 130a, 130b, 130c bears on the outer surface 122 of the object 2, the device 1 thus has at least three separate bearing zones (corresponding respectively to the three carriages 130a, 130b, 130c) in the longitudinal direction D. The device 1 is therefore positioned stably relative to the object 2 and the distance between the primary magnetic sensor 12 and the object 2 remains constant during the use of the device 1.

The three carriages 130a, 130b, 130c comprise a primary carriage 130a, a secondary carriage 130b and a tertiary carriage 130c.

The primary carriage 130a holds the magnetic field generator 10 and the primary magnetic sensor 12. The primary carriage 130a also preferably holds at least one stabilizing element 18a, referred to as the “primary stabilizing element”.

Preferably, as illustrated in FIG. 9, the primary stabilizing element 18a is a roller, referred to as the “primary roller”, the axis of rotation Xa of which is perpendicular to the longitudinal direction D. Consequently, the primary stabilizing element 18a allows the primary carriage 130a to roll on the outer surface 22 of the object 2 while maintaining the magnetic field generator 10 and the primary magnetic sensor 12 at a constant, predefined distance from the outer surface 22.

The secondary carriage 130b holds the secondary magnetic sensor 16 and at least one secondary stabilizing element 18b. The secondary stabilizing element 18b is preferably a roller referred to as the “secondary roller” the axis of rotation Xb of which is perpendicular to the longitudinal direction (D). The secondary roller 18b allows the secondary carriage 130b to roll on the outer surface 22 while maintaining the secondary magnetic sensor 16 at a constant, predefined distance from the outer surface 22.

The tertiary carriage 130c holds at least one tertiary stabilizing element 19c. The tertiary stabilizing element 19c is preferably a roller referred to as the “tertiary roller” the axis of rotation Xc of which is perpendicular to the longitudinal direction D. The tertiary roller 19c allows the tertiary carriage 130c to roll on the outer surface 22.

Preferably, the carriages 130a, 130b, 130c are connected to each other by pivot connections, typically in pitch.

As illustrated in FIG. 9, the device 1 comprises at least one suspension member 19 for stressing at least one carriage 130a, 130b, 130c (in FIG. 9, the primary carriage 130a) so as to press at least one carriage 130a, 130b, 130c against the object 2 when the device 1 is in use.

As explained above, the carriages 130a, 130b, 130c are placed in series. The carriages 130a, 130b, 130c therefore comprise a front carriage, a central carriage and a rear carriage, the front carriage being the first in the line of carriages 130a, 130b, 130c in a primary sense S in the longitudinal direction D as illustrated in FIG. 9. In use, during a relative movement of the device 1 with respect to the object 2, the same zone of the object 2 is first facing the front carriage, then the central carriage and finally the rear carriage.

Preferably, the front carriage corresponds to the secondary carriage 130b, the central carriage corresponds to the primary carriage 130a and the rear carriage corresponds to the tertiary carriage 130c.

The front carriage, the central carriage and/or the rear carriage can be stressed by a suspension member 19.

The device 1 also comprises a processing unit such as a processor. The processing unit is capable of acquiring and analysing signals received by the primary magnetic sensor 12 and the secondary magnetic sensor 16.

### Assembly

As explained above, the device 1 is capable of being moved relative to the object 2. A detection assembly is thus proposed, comprising the device 1 and at least one movement system capable of allowing relative movement of the device 1 with respect to the object 2.

According to a preferred embodiment, the movement system makes it possible to cause the outer surface 22 of the object 2 to travel facing the magnetic field generator 10 and the primary magnetic sensor 12 in a predetermined sense B referred to as the “scanning sense”, as illustrated in FIG. 1. Here, “travel” is given to mean a relative movement of the outer surface 22 with respect to the device 1, and is not limited to the movement of the outer surface. The travelling can be made possible by moving the device 1 relative to the outer surface 22 of the object 2.

The movement system can be a robotic arm. Preferably, the device 1 is capable of being attached to a robotic arm. The robotic arm is capable of moving the device 1 and holding it stably in a certain position facing the object 2.

According to a preferred embodiment, the object 2, which is typically a ply of a tyre, is stretched around a drum and the movement system is a drive system that rotates the drum so that the object 2 is rotated and the outer surface 22 thereof travels facing the device 1. Conversely, the drum can be fixed and the drive system can rotate the device 1 relative to the drum.

Preferably, the scanning sense B is such that the secondary magnetic sensor 16 is arranged so that it precedes the primary magnetic sensor 14 with respect to the scanning sense. In other words, the secondary carriage 130b is arranged so that it precedes the primary carriage 130a with respect to the scanning sense. The movement system is thus designed so that, in use, the same zone of the outer surface 22 of the object 2 is successively covered by the secondary magnetic sensor 16 and then by the primary magnetic sensor 12.

### Method

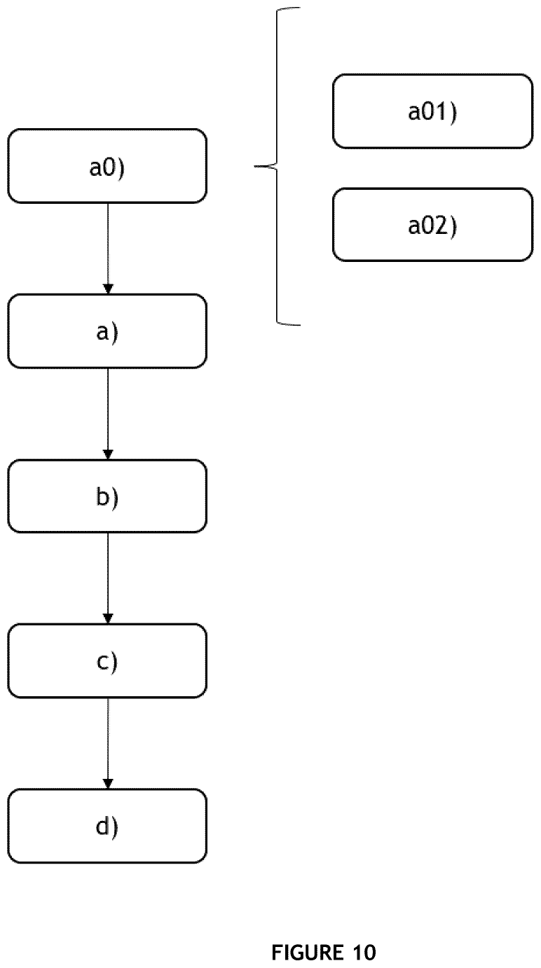

With reference to FIG. 10, a method is proposed for detecting, in an object containing a plurality of metal cords 20, a positioning defect of one of the metal cords 20 relative to another of the metal cords 20 using the device 1.

Advantageously, the method is implemented in order to detect, in a reinforcing ply, for example a carcass ply of a tyre, containing metal cords 20 extending non-parallel to a selvedge of the reinforcing ply, a positioning defect of one of the metal cords 20 relative to another of the metal cords 20.

The method preferably comprises a step a0) of positioning the primary magnetic sensor 12 relative to the magnetic field generator 10 so that the primary magnetic sensor 12 is placed in a chosen position that corresponds to the position of a neutral point of the inspection magnetic field, i.e. the magnetic field emitted by the magnetic field generator 10. Step a0) is a step of adjusting the device 1.

To this end, step a0) preferably comprises a step a01) of the processing unit acquiring a no-load signal received by the primary magnetic sensor 12 when it is not subject to a magnetic field, in particular the inspection magnetic field. In other words, the magnetic field intensity, referred to as the “no-load magnetic field”, is measured when the primary magnetic sensor 12 is not near any magnetic disturbance. Then, step a0) preferably comprises a step a02) of placing the primary magnetic sensor 12 relative to the magnetic field generator 10 so that the signal received by the primary magnetic sensor 12 is equal to the no-load signal. It is thus accepted that the primary magnetic sensor 12 is placed at a neutral point of the inspection magnetic field when it measures a no-load signal while being in the inspection magnetic field.

Step a02) comprises moving the primary magnetic sensor 12 relative to the current generator 10. In particular, according to a certain embodiment, the second primary support part 142 that holds the primary magnetic sensor 12 is translated along the central axis of the generator A relative to the first primary support part 141 that holds the magnetic field generator 10, for example by screwing/unscrewing.

Preferably, as explained above, the central axis of the generator A corresponds to the axis connecting the north pole and the south pole of the magnetic field generator 10. Consequently, it will be understood that translating the second primary support part 142 along the central axis of the generator A makes it possible to easily position the primary magnetic sensor 12 at a neutral point of the inspection magnetic field.

The method comprises a step a) of supplying the device 1 adjusted in this way.

The method then comprises a step b) of positioning the device 1 relative to the object 2 so that the magnetic field generator 10 and the primary magnetic sensor 12 are positioned facing the object 2, more specifically facing the outer surface of the object 2.

Preferably, the device 1 is positioned relative to the object 2 so that the axis connecting the north pole and the south pole of the magnetic field generator 10 is perpendicular to a plane tangent to the outer surface 22 of the object 2.

It will be understood that the magnetic field is free. In other words, the magnetic field is not guided as it could be for example by branches capable of allowing the circulation of the magnetic field.

The device 1 is preferably placed so that the stabilizing element or elements 18 of the device 1 are in contact with the object 2. Consequently, the distance between the object 2 and the magnetic field generator 10 and between the object 2 and the primary magnetic sensor 12 is known and is considered to be constant provided that the stabilizing elements 18 of the device 1 are in contact with the object 2.

When the device 1 is positioned in this way, the inspection magnetic field undergoes deformation due to the metal cords 20 of the object 2. Here, the device 1 is considered to not be placed facing a defect of the object 2. In other words, the device 1 is not placed facing a zone of the object 2 that is likely to have a defect, such as for example a weld zone between two edges of a metal reinforcing ply of a tyre, which is referred to as the “zone of interest”.

Preferably, initially, and therefore during the implementation of step b), the device 1 is thus positioned so that it is not facing a defect of the object 2.

In this position, the primary magnetic sensor 12 is considered to be in a position referred to as the “nominal position” and receives a signal referred to as the “nominal signal” that has a magnetic field intensity referred to as the “nominal intensity”. In other words, when the primary magnetic sensor 12 is positioned facing the object 2 while not being positioned facing a defect, it receives a nominal signal that results from the deformations of the inspection magnetic field due solely to the interference between the inspection magnetic field and the metal cords 20 of the object 2. This nominal signal is in fact a base signal and indicates the signal that is supposed to be received by the primary magnetic sensor 12 when no defect is detected in the object 2.

According to one embodiment, step b) also comprises positioning the secondary magnetic sensor 16 facing the object 2. This comprises for example placing the stabilizing element 18b in contact with the secondary carriage 130b holding the secondary magnetic sensor 16 so that the magnetic sensor is placed at a known distance from the object 2.

According to one embodiment, step b) comprises placing the sub-assembly 13 comprising the train of three carriages 130a, 130b, 130c placed in series in the longitudinal direction D. The carriages 130a, 130b, 130c are positioned so that the longitudinal direction D is collinear with the planned direction of movement of the device 1 relative to the object 2. Advantageously, the carriages 130a, 130b, 130c are positioned so that the longitudinal direction D is collinear with the planned sense of scanning of the outer surface 22 the object 2. In other words, the carriages 130a, 130b, 130c are positioned so that they are in series in the scanning sense. Preferably, they are positioned so that the front carriage is the secondary carriage 130b, the central carriage is the primary carriage 130a and the rear carriage is the tertiary carriage 130c.

Step b) can for example be implemented by means of the robotic arm 3 that handles the device 1.

The method then comprises a step c) of moving the device 1 relative to the object 2. According to one embodiment, the device 1 is moved, for example by the robotic arm, while the object 2 remains immobile. According to another embodiment, the object 2 is set in motion relative to the device 1, while the device 1 is for example held immobile by the robotic arm.

Preferably, the object 2 is wrapped around a drum and is capable of being rotated about its axis of rotation by the movement system, which is a drive system. As a result, the outer surface 22 of the object 2 travels facing the magnetic field generator 10 and the primary magnetic sensor 12 in the scanning sense. Preferably, the drive system drives the object 2 so that the object 2 only performs a single complete rotation. Conversely, the drive system can drive the magnetic field generator and the primary magnetic sensor so that they travel relative to the drum.

It will therefore be understood that, during the relative movement, each zone of the object 2 that travels facing the magnetic field generator 10 is subjected to the inspection magnetic field.

According to a certain embodiment, as explained, preferably, during the relative movement, the same zone of the outer surface 22 of the object 2 is successively covered by the secondary magnetic sensor 16 and then by the primary magnetic sensor 12. When a zone of the object 2 is facing the secondary magnetic sensor 16, said zone thus has advantageously not been magnetized by the magnetic field generator 10. As a result, the secondary magnetic sensor 16 receives a secondary signal that corresponds to the background magnetic field, i.e. to a residual magnetic field of the object 2 and/or a magnetic field of the environment, and this secondary signal is advantageously independent of any magnetization of the object 2 due to the magnetic field generator 10.

According to the embodiment in which the device 1 comprises a secondary magnetic sensor 16, step c) comprises the processing unit continuously acquiring a secondary signal received by the secondary magnetic sensor 16. The secondary signal corresponds to the change in the intensity of the background magnetic field during the relative movement.

Step c) further comprises the processing unit continuously acquiring a primary signal received by the primary magnetic sensor 12. The primary signal varies in particular as a function of the variations in the intensity of the inspection magnetic field during the relative movement.

Preferably, during the relative movement, the primary magnetic sensor 12 is placed successively in different positions relative to the object 2 and the different positions comprise at least one position of interest in which the primary magnetic sensor 12 is placed facing a zone of interest of the object 2 and a nominal position. As explained above, the zone of interest corresponds to a zone of the object 2 likely to have a defect, i.e. a zone in which a potential defect is to be detected. The nominal position corresponds to a position in which the primary magnetic sensor 12 is not placed facing a defect of the object 2. In other words, it is desirable for the device 1 to be placed facing the object 2 so that it is positioned facing at least one zone of interest and at least one zone without defects. Being positioned facing at least one zone with defects makes it possible to determine a nominal magnetic field intensity of the primary signal in order to be able to detect a variation in intensity of the primary signal relative to the nominal intensity. Being positioned facing at least one zone of interest makes it possible to acquire a primary signal in order to detect the presence or absence of a defect in this zone of interest.

The method comprises a step d) of detecting, in the primary signal, a signature representative of a positioning defect of a metal cord 20 of the object 2. Step d) is preferably implemented by the processing unit. Step d) preferably comprises the processing unit analysing and processing the primary signal.

Preferably, step d) comprises processing of the primary signal that includes subtracting the secondary signal received by the secondary magnetic sensor 16 from the primary signal received by the primary magnetic sensor 12. This makes it possible to clean the primary signal by subtracting from it the variations in magnetic field intensity linked to the background magnetic field.

Step d) can comprise different processing operations in order to facilitate the analysis of the primary signal. Step d) can therefore comprise at least one processing operation among, for example, the following processing operations: filtering with a low-pass filter, calculation of the absolute value of the signal, normalization, and thresholding. These different operations can make it possible for example to eliminate background noise or to flatten the signal and thus make the signal clearer and easy to interpret. A combination of these operations can also be implemented in order to process the primary signal.

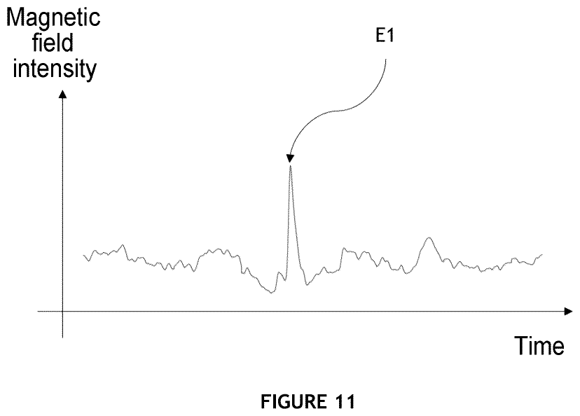

The signature representative of a positioning defect of a metal cord of the object is a local extremum of the primary signal. The local extremum corresponds to a local maximum of the primary signal that is greater than a first threshold value, or to a local minimum of the primary signal that is less than a second threshold value. For example, FIG. 11, which shows a primary signal, i.e. the change in the magnetic field intensity measured by the primary magnetic sensor 12 as a function of time, illustrates a local extremum E1 corresponding to a local maximum of the primary signal. The first and second threshold values are defined relative to the nominal signal. For example, the measured magnetic field intensity corresponding to the first threshold value can correspond to an intensity that is greater than the nominal intensity by a certain difference. Likewise, for example, the measured magnetic field intensity corresponding to the second threshold value can correspond to an intensity that is less than the nominal intensity by a certain difference. It will be understood that the first threshold value is greater than the nominal intensity and the second threshold value is less than the nominal intensity.

The presence of a local extremum indicates a variation between the magnetic field intensity of the primary signal and the nominal intensity, i.e. the magnetic field intensity of the primary signal that corresponds to the magnetic field intensity when the magnetic field generator 10 is not placed facing a defect of the object 2. This indicates the deformation of the inspection magnetic field. The deformation of the inspection magnetic field indicates a potential defect of the object 2.

Advantageously, if the presence of a local extremum is determined and, if this local extremum has a magnetic field intensity greater than the first threshold value, it is concluded that there is a defect corresponding to a superposition of two metal cords 20. However, if the presence of a local extremum is determined and, if this local extremum has a magnetic field intensity less than the second threshold value, it is concluded that there is a defect corresponding to the existence of a gap between two consecutive metal cords 20 that is greater than a nominal gap.

According to a certain embodiment, the absolute value of the magnetic field intensity of the primary signal is used (it is accepted that processing making it possible to calculate the absolute value of the signal is implemented), which can facilitate the analysis of the primary signal. In this case, it will be understood that all of the values of the primary signal processed are positive.

If a defect is detected, various actions can be implemented. For example, the object 2 can be replaced correctly or modified so as to remove the defect.

The invention is not limited to the embodiment described and shown in the appended figures. Modifications are possible, in particular from the point of view of the composition of the various technical features or by substituting technical equivalents, without however departing from the general scope of the invention.

1.-22. (canceled)

23. A detection device for detecting, in an object containing a plurality of metal cords, a positioning defect of one of the metal cords relative to another of the metal cords, the detection device comprising:
a magnetic field generator, that is arranged to generate an inspection magnetic field and to be positioned facing the object so that the plurality of metal cords causes deformation of the inspection magnetic field; and
a primary magnetic sensor that is arranged to measure an intensity of the inspection magnetic field,
wherein the inspection magnetic field has, before being deformed by the plurality of metal cords, at least one neutral point at which the intensity of the inspection magnetic field is zero, and
wherein the primary magnetic sensor is placed relative to the magnetic field generator in a chosen position that corresponds to a position of the at least one neutral point.

24. The detection device according to claim 23, further comprising a primary support that holds the magnetic field generator and the primary magnetic sensor and is provided with an adjustment system making it possible to adjust, a position of the primary magnetic sensor relative to the magnetic field generator, in order to reach the chosen position, and to hold the primary magnetic sensor in the chosen position, fixed relative to the magnetic field generator.

25. The detection device according to claim 24, wherein the magnetic field generator is a toroid that is centered on a central axis of the magnetic field generator and defines a free inner space, and
wherein the adjustment system comprises a support part that holds the primary magnetic sensor and is engaged in the free inner space so that it can be moved along the central axis of the magnetic field generator in order to modify the position of the primary magnetic sensor along the central axis of the magnetic field generator.

26. The detection device according to claim 23, further comprising at least one stabilizing element, capable of coming into contact with the object in order to maintain a constant distance between the primary magnetic sensor and an outer surface of the object.

27. The detection device according to claim 26, further comprising at least one suspension member, capable of elastically pressing the stabilizing element against the object.

28. The detection device according to claim 23, further comprising a secondary magnetic sensor that is separate from the primary magnetic sensor, is not subject to the inspection magnetic field, and is arranged to be placed facing a zone of the object in order to measure an intensity of a background magnetic field, which is representative of a magnetic field prevailing in the object in absence of the inspection magnetic field.

29. The detection device according to claim 28, further comprising a sub-assembly forming a train of three carriages that are placed in series in a longitudinal direction and are articulated in pairs, one relative to the other, so that they are able to follow an outer surface of the object, each bearing on the outer surface in order to provide at least three corresponding separate bearing zones along the longitudinal direction, the carriages respectively being made up of:
a primary carriage that holds the magnetic field generator and the primary magnetic sensor, together with at least one primary stabilizing element, which allows the primary carriage to roll on the outer surface of the object while maintaining the magnetic field generator and the primary magnetic sensor at a constant, predefined distance from the outer surface;
a secondary carriage that holds the secondary magnetic sensor and at least one secondary stabilizing element, which allows the secondary carriage to roll on the outer surface while maintaining the secondary magnetic sensor at a constant, predefined distance from the outer surface; and
a tertiary carriage that holds at least one tertiary stabilizing element, which allows the tertiary carriage to roll on the outer surface of the object.

30. A detection assembly comprising the detection device according to claim 28, together with a drive system, making it possible to generate a relative travelling movement of an outer surface of the object with respect to the magnetic field generator and the primary magnetic sensor in a predetermined scanning sense, and
wherein the secondary magnetic sensor is arranged so as to precede the primary magnetic sensor with respect to the scanning sense, so that a same zone of the outer surface of the object is covered successively by the secondary magnetic sensor, which measures the background magnetic field, and then by the primary magnetic sensor, which measures the inspection magnetic field as deformed by the plurality of metal cords of the object.

31. A method for detecting, in an object containing a plurality of metal cords, a positioning defect of one of the metal cords relative to another of the metal cords, the method comprising the following steps:
(a) supplying a magnetic field generator and a primary magnetic sensor, in order to form a detection device, the primary magnetic sensor being placed relative to the magnetic field generator in a chosen position that corresponds to a position of a neutral point, the neutral point corresponding to a point of an inspection magnetic field generated by the magnetic field generator, before it is deformed by the plurality of metal cords, at which an intensity of the inspection magnetic field is zero;
(b) positioning the detection device relative to the object so that the magnetic field generator and the primary magnetic sensor are placed facing the object;
(c) moving the detection device relative to the object and continuously acquiring a primary signal received by the primary magnetic sensor; and
(d) detecting, in the primary signal, a signature representative of a positioning defect of a metal cord of the object.

32. The method according to claim 31, wherein step (a) comprises a step (a0) of adjusting the detection device by positioning the primary magnetic sensor relative to the magnetic field generator in the chosen position.

33. The method according to claim 32, wherein, in step (a0), the primary magnetic sensor is positioned using an adjustment system that makes it possible to adjust a position of the primary magnetic sensor relative to the magnetic field generator and to keep the primary magnetic sensor in the chosen position, fixed relative to the magnetic field generator, the adjustment system being contained in a primary support, which holds the magnetic field generator and the primary magnetic sensor and is provided with the adjustment system.

34. The method according to claim 33, wherein the magnetic field generator is a toroid centered on a central axis of the magnetic field generator and defines a free inner space, and the adjustment system comprises a support part that holds the primary magnetic sensor and is engaged in the free inner space, and
wherein, in step (a0), the support part holding the primary magnetic sensor is moved along the central axis of the magnetic field generator in order to modify the position of the primary magnetic sensor along the central axis of the generator.

35. The method according to claim 31, wherein, in step (b), at least one stabilizing element is placed in contact with the object in order to maintain a constant distance between the primary magnetic sensor and an outer surface of the object.

36. The method according to claim 35, wherein, in step (b), the stabilizing element is elastically pressed against the object by a suspension member.

37. The method according to claim 31, wherein, during step (c), the detection device is moved relative to the object so that the primary magnetic sensor is placed successively in different positions relative to the object, the different positions comprising at least one position of interest, wherein the magnetic sensor is placed facing a zone of interest of the object, wherein a potential defect is to be detected, and a nominal position, wherein the magnetic sensor is placed facing a zone of the object without defects, different from the zone of interest, so that a nominal magnetic field intensity of the primary signal is measured.

38. The method according to claim 37, wherein a signature representative of a positioning defect of a metal cord of the object is a local extremum relative to a nominal magnetic field intensity, the local extremum having a magnetic field intensity greater than a first threshold value or a magnetic field intensity less than a second threshold value, and
wherein, in step (d), when presence of a local extremum is determined and, when this extremum has a magnetic field intensity greater than the first threshold value, it is concluded that a defect is present corresponding to the superposition of two metal cords, whereas, when this extremum has a magnetic field intensity less than the second threshold value, it is concluded that a defect is present corresponding to an existence of a gap between two consecutive metal cords that is greater than a nominal gap.

39. The method according to claim 31, comprising a step, prior to acquiring the primary signal, of acquiring a secondary signal corresponding to a change during a movement of a magnetic field referred to as a “background magnetic field”, which is representative of a magnetic field prevailing in the object in the absence of the inspection magnetic field, and wherein step d) comprises a processing of the primary signal including the subtraction of the secondary signal from the primary signal, and wherein the step of acquiring a secondary signal is implemented by a secondary magnetic sensor that is separate from the primary magnetic sensor and is not subject to the inspection magnetic field and, during the implementation of the step of acquiring a secondary signal, the secondary magnetic sensor is placed facing a zone of the object.

40. The method according to claim 39, wherein, in step (b), a sub-assembly is placed in contact with an outer surface of the object, the sub-assembly forming a train of three carriages that are placed in series in a longitudinal direction and are articulated in pairs, one relative to the other, so that they are able to follow an outer surface of the object, each bearing on the outer surface in order to provide at least three corresponding separate bearing zones along the longitudinal direction, the carriages respectively being made up of:
a primary carriage that holds the magnetic field generator and the primary magnetic sensor, together with at least one primary stabilizing element, which allows the primary carriage to roll on the outer surface of the object while maintaining the magnetic field generator and the primary magnetic sensor at a constant, predefined distance from the outer surface;
a secondary carriage that holds the secondary magnetic sensor and at least one secondary stabilizing element, which allows the secondary carriage to roll on the outer surface while maintaining the secondary magnetic sensor at a constant, predefined distance from the outer surface; and
a tertiary carriage that holds at least one tertiary stabilizing element, which allows the tertiary carriage to roll on the outer surface of the object, and
wherein, in order to implement the step of acquiring a secondary signal and the step (c) of moving the detection device relative to the object and continuously acquiring a primary signal received by the primary magnetic sensor, the carriages are rolled on the outer surface of the object so that a same zone of the object is first facing the primary carriage, then the secondary carriage and finally the tertiary carriage.

41. The method according to claim 31, wherein the step (d) comprises a processing of the primary signal, the processing comprising at least one processing operation among the following processing operations: a filtering with a low-pass filter, calculation of an absolute value of the primary signal, a normalization, and a thresholding.

42. The method according to claim 31, the method being implemented in order to detect, in a reinforcing ply containing metal cords extending non-parallel to a selvedge of the reinforcing ply, a positioning defect of one of the metal cords relative to another of the metal cords.
